(19) United States
(12) Patent Application Publication
(10) Pub. No.: US 2021/0310979 A1
(43) Pub. Date: Oct. 7, 2021

(54) PROBE ARRAYS

(71) Applicant: 3BRAIN AG, Wadenswil (CH)

(72) Inventors: Mauro Gandolfo, Zurich (SE); Alessandro Maccione, Genova (IT); Kilian Imfeld, Wadenswil (CH); Francis Cardot, Neuchatel (CH)

(21) Appl. No.: 17/268,894

(22) PCT Filed: Aug. 15, 2019
(86) PCT No.: PCT/EP2019/071960

§ 371 Date: Feb. 16, 2021

(30) Foreign Application Priority Data
Aug. 17, 2018 (GB) 1813457.7

(51) Int. Cl. G01N 27/327; G01N 33/483

(52) U.S. Cl. CPC G01N 27/3272; G01N 33/4836

(57) ABSTRACT

A probe array comprises a substrate having an array of pillar probes which may be electrodes. Each pillar probe extends in a direction orthogonal to the plane of the substrate and has a first cross-sectional area. Each probe is disposed on a pedestal formed on the substrate where each pedestal has a second cross-sectional area greater than the first cross-sectional area and forms a platform from which the pillar probe extends, the platforms configured to support the underside of a sample structure which has been penetrated by the pillar probes to thereby prevent the sample structure from reaching the substrate. A plurality of channels is formed between the pedestals, the channels each having a width less than the spacing between the pillar probes. The channels ensure that a supply of critical fluids can be delivered to an underside of a cellular sample into which the pillar probes penetrate.

The invention relates to probe arrays and in particular though not exclusively, to probe arrays suitable for use in neural recording systems.

The brain is a network of a large number of different cells that are in constant cell-to-cell communication. Each of the 100 billion neurons in the brain is connected with approximately 10000 other neurons by means of axons, which carry action potentials (AP)—electrical signals—to communicate with other cells. Any sensorimotor information, like moving an arm or tasting something bitter, any emotion, thought or mental cognition is ultimately coded into a complex firing pattern of APs involving many cells. The more complex the task, the more cells are involved. Understanding how signals are processed in the brain in health and in disease is an area of strong scientific and medical interest. Techniques currently applied are electro-physical: in vitro models (i.e. biological preparations of cells in a Petri dish) provide better accessibility and a controlled environment, whereas in vivo models (animals) present a much higher challenge. Due to the heterogeneous cellular composition of the brain, a high number of cells need to be measured at the same time to gain any relevant understanding of the brain's activities.

Existing systems utilise micromachined electrode arrays (MEAs) to provide a large number of electrodes by which electrical communication with the cells can take place. As shown schematically in FIG. 1, such MEAs 1 may be fabricated on a suitable substrate 2 such as a glass-based or silicon-based substrate, and comprise conductive paths 3 and electrodes 4 deposited on the substrate 2. Each electrode 4 comprises a region of electrically conductive metal that is electrically connected by, for example a thin metal wire or deposited track 3 to a respective connector pad 5 also disposed on the substrate 2. The connector pads 5 may be disposed around the periphery of the substrate 2 to facilitate electrical connection of each electrode 4 to external circuitry. The electrodes 4 may be brought into contact with cellular material such as brain tissue under analysis to read activity from cells by amplifying and converting to digital signals by means of analogue-to-digital converters.

The electrodes 4 can be used both for recording signals from cells and for stimulating cells by releasing small injections of current, thus represent a bidirectional communication system with the cells.

Some MEAs 1 may include on-board electronics fabricated on the substrate 2, e.g. in layers under the array of electrodes 4 using, for example, CMOS technology. The CMOS technology may integrate signal amplification stages onto the MEA (e.g. for improving signal to noise ratio) and/or switching structures that allow multiplexing of high density electrodes on the array by connecting and disconnecting distinct groups of the electrodes, thus limiting the number of wires necessary to electrically address all the electrodes.

The electrodes 4 may typically be 5 to 30 μm in diameter or on the side in case of rectangular shaped electrodes and have a pitch or a separation distance ranging approximately from 15 to 200 μm. Throughout the present specification, the expression ‘pitch’ is used in the normal sense of the distance from point-to-corresponding-point of a repeating structure, e.g. from point-to-point of an electrode array, whereas the expression ‘separation’ is used to indicate the distance between two structures, e.g. the distance between two electrode structures.

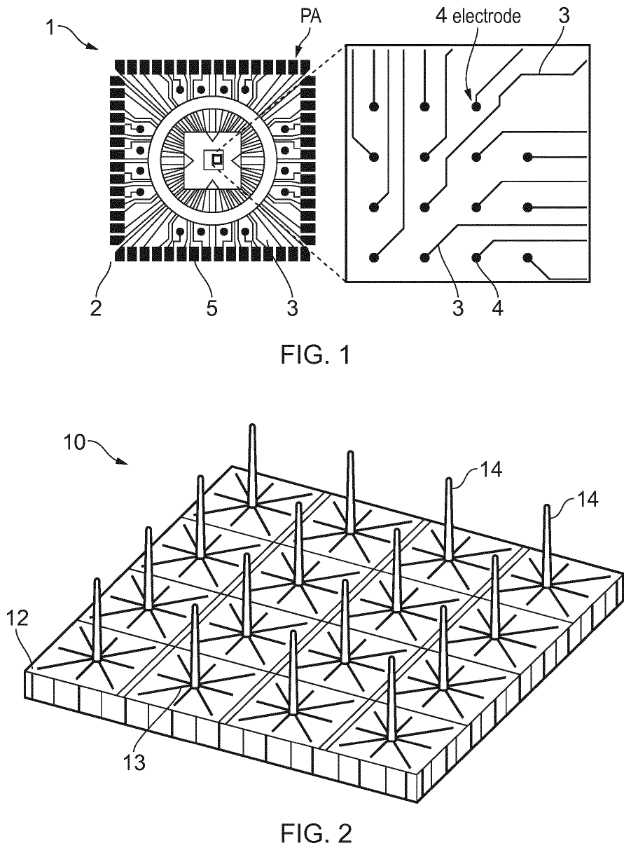

Earlier MEAs, as described above, comprise planar electrodes. More recently, developments in MEAs include so-called ‘3D electrodes’ which comprise an array of needles extending orthogonally from the plane of the substrate which can penetrate for instance a tissue sample slice disposed thereon, thereby making better electrical connections within the tissue sample under analysis and establishing an electrical connection with deeper layers of the tissue. An example of a 3D electrode MEA is shown in FIG. 2. In a 3D electrode array 10, the substrate 12 and conductive paths 13 and other connection arrangements, may be similar to those described for the MEA 1 of FIG. 1, but the electrodes 14 are each formed as a needle extending perpendicularly from the substrate 12. In some examples, the needles may have diameters in the range 5 to 100 microns, and may have a length (i.e. height above the substrate) in the range 50 to 2000 microns. In some MEAs, each needle may have multiple, separately addressable electrodes on each needle.

One issue that has been noted to arise with existing planar and 3D MEAs is that tissue structures may suffer from restricted or completely occluded flows of critical or essential fluids to the tissue structures on the side that is facing the MEA substrate 12. This can be particularly relevant for in vitro analysis of thick structures, such as three-dimensional cell preparations (e.g. neuronal or cardiac) developed with hydrogels, membranes or other types of scaffolding, and three-dimensional tissue samples such as thick brain tissue, organoids, and dense neuronal assemblies.

Free flowing fluid access to the top surface of such structures under analysis may be possible from above the MEA needle array, but access to a lower surface of the structures may be significantly restricted or prevented by the substrate 12.

It is an object of the invention to provide improvements in microelectrode arrays which may assist in overcoming such problems.

According to one aspect, the present invention provides a probe array comprising:
a substrate having an array of pillar probes, each pillar probe extending in a direction orthogonal to the plane of the substrate and having a first cross-sectional area, each probe being disposed on a pedestal formed on the substrate wherein each pedestal has a second cross-sectional area greater than the first cross-sectional area and forms a platform from which the pillar probe extends,
wherein a plurality of channels is formed between the pedestals, the channels each having a width less than the spacing between the pillar probes.

Each pedestal may define an exposed surface substantially parallel to the plane of the substrate at the base of the pillar extending therefrom. The pedestals may provide a plurality of said exposed surfaces configured to provide a platform for supporting the underside of a sample structure when penetrated by the pillar probes to thereby prevent the sample structure from reaching the substrate. The pedestal height may be greater than 10 microns. The channels may each have a width greater than 10 microns. The channels may each have a width greater than 14 microns. The pedestals may define the channels to have a cross-sectional area of at least 100 square microns for flow of fluids therethrough when a sample structure is penetrated by the pillar probes and supported on the platform defined by the pedestal. The pedestals may define the channels to have a cross-sectional area of at least 140 square microns for flow of fluids therethrough when a sample structure is penetrated by the pillar probes and supported on the platform defined by the pedestal. The probe array may further include a plurality of pedestals each having no pillar probes disposed thereon. At least some pedestals may each have a plurality of pillars disposed thereon. The pedestals and pillars may be arrayed in a two-dimensional grid area with a first set of channels extending in a first direction along the plane of the substrate between the pedestals and a second set of channels extending in a second direction along the plane of the substrate between the pedestals. The pedestals may be rectangular in cross-section and the first set of channels are orthogonal to the second set of channels. The pillars may be arrayed in a two-dimensional grid area with the pedestals forming a one-dimensional array with channels therebetween extending across the two-dimensional grid area in one direction, each pedestal supporting a plurality of pillars. The probe array may include a plurality of supply channels each supply channel extending parallel to the substrate outside the two-dimensional grid area and communicating with at least one of the channels within the two-dimensional grid area. The probe array may include a first plurality of supply channels each extending parallel to the substrate outside the two-dimensional grid area in the first direction and communicating with at least one of the first set of channels. The probe array may include a second plurality of supply channels each extending parallel to the substrate outside the two-dimensional grid area in the second direction and communicating with at least one of the second set of channels. Some or all of the probes in an array may comprise electrodes. The probes may comprise pillar electrodes and may further comprise an electrically insulating material extending over a major portion of the surfaces of the pedestals and pillar electrodes. At least some probes may comprise an optical sensor and/or an optical actuator. The pedestals and pillars may be both formed of the same electrically conductive material. The electrically conductive material may comprise at least one of gold, platinum, iridium and alloys thereof. The pedestals and at least a lower portion of each pillar may be coated with an electrically insulating material. Each probe may be electrically isolated from other probes and electrically connected to a respective connector pad on the substrate or a respective integrated circuit disposed on or in the substrate.

According to another aspect, the invention provides a method of fabricating a probe array comprising:
forming a plurality of pedestals onto a substrate, the pedestals being separated along at least one axis to form a plurality of channels between the pedestals, the channels extending along an axis parallel to the surface of the substrate and transverse to the axis of separation of the pedestals;
forming a plurality of pillar probes on the pedestals, each pillar extending in a direction orthogonal to the plane of the substrate and having a first cross-sectional area,
wherein each pedestal has a second cross-sectional area greater than the first cross-sectional area and forms a platform from which the pillar probe extends, and wherein each of the channels has a width less than the spacing between the pillar probes.

The method may further comprise the steps of:
forming a first photoresist layer on the substrate to a first depth and defining a first plurality of openings therein;
depositing a first metallic layer in the first plurality of openings to form the pedestals;
forming a second photoresist layer on the first metallic layer and on any residual parts of the first photoresist layer, and defining a second plurality of openings in the second photoresist layer that are co-registered with the first metallic layer within the first plurality of openings, each of the second plurality of openings being smaller in cross-sectional area than each of the first plurality of openings;
depositing a second metallic layer in the second plurality of openings to form the pillar probes;
removing any residual first and second photoresist layers.

The pedestals and pillars may be formed of gold. The deposition of one or both of the first and second metallic layers may comprise an electroplating process. The method may further include, prior to forming the plurality of pedestals on the substrate, forming an oxide layer on the substrate. The method may further include depositing an electrically insulating layer onto a major portion of the pillars and pedestals. Depositing an electrically insulating layer may comprise depositing the electrically insulating layer to cover the pillar and pedestal structures and selectively removing parts of the electrically insulating layer on the top and/or sides of at least some of the pillars.

Embodiments of the present invention will now be described by way of example and with reference to the accompanying drawings in which:

FIG. 1 shows a schematic view of an example of a prior art micro-electrode array (MEA) device;

FIG. 2 shows a perspective view of an example of a prior art 3D MEA device;

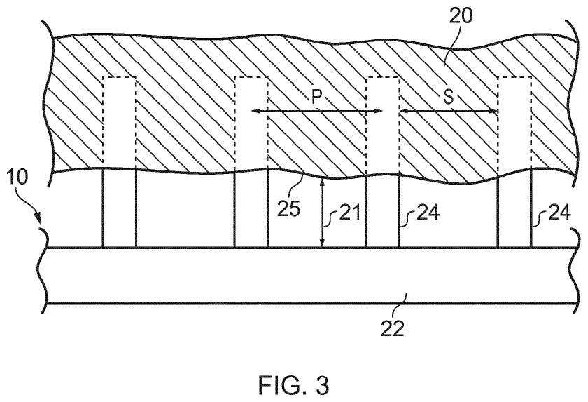

FIG. 3 shows a schematic cross-sectional view of a 3D MEA device engaged with a sample structure under analysis;

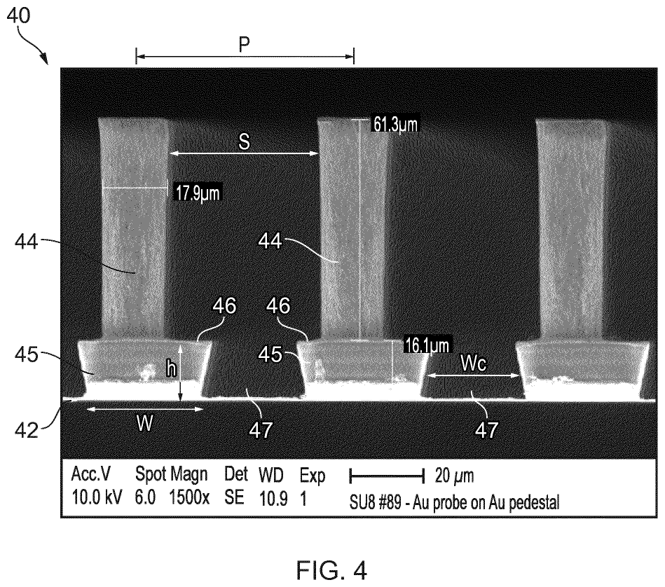

FIG. 4 shows a scanning electron micrograph of a cross-section of an electrode structure having pillars and pedestals, in a micro-electrode array;

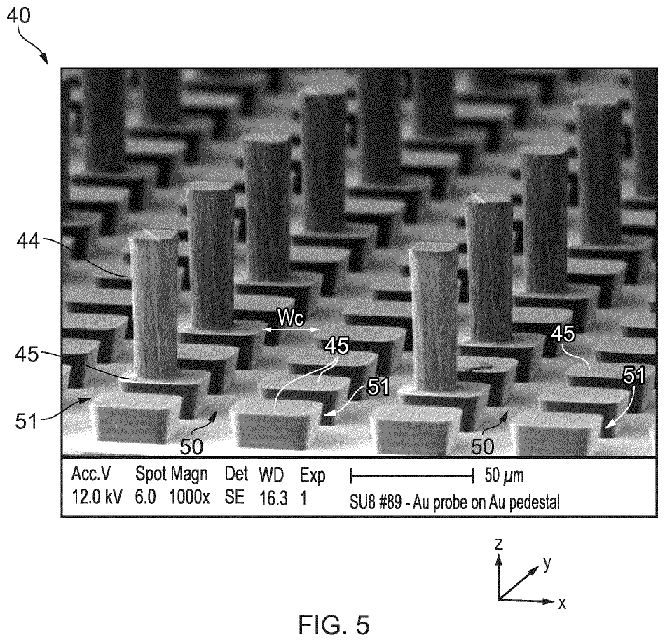

FIG. 5 shows a scanning electron micrograph of an array of electrodes such as those shown in FIG. 4 in a microelectrode array;

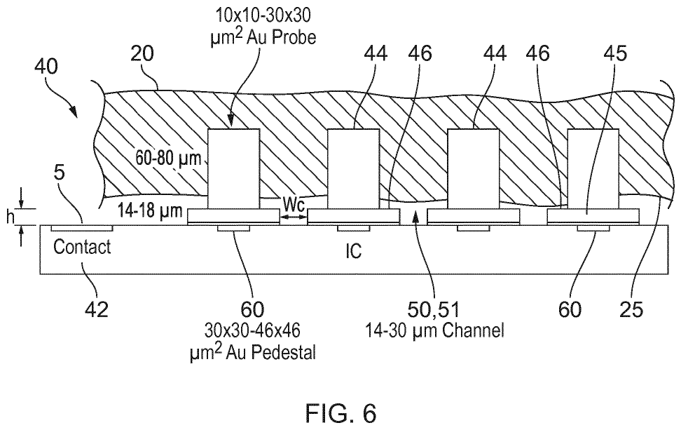

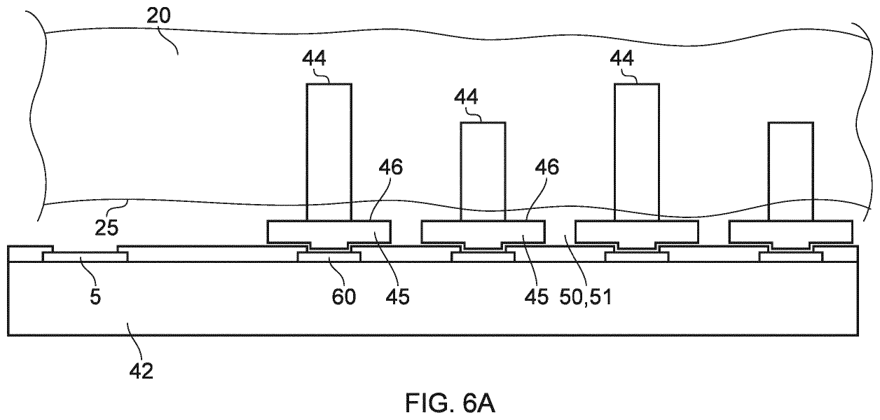

FIG. 6 is a schematic cross-sectional view of a monolithic integrated circuit incorporating the microelectrode array of FIG. 5;

FIG. 6A is a schematic cross-sectional view of a microelectrode array with varying pillar heights;

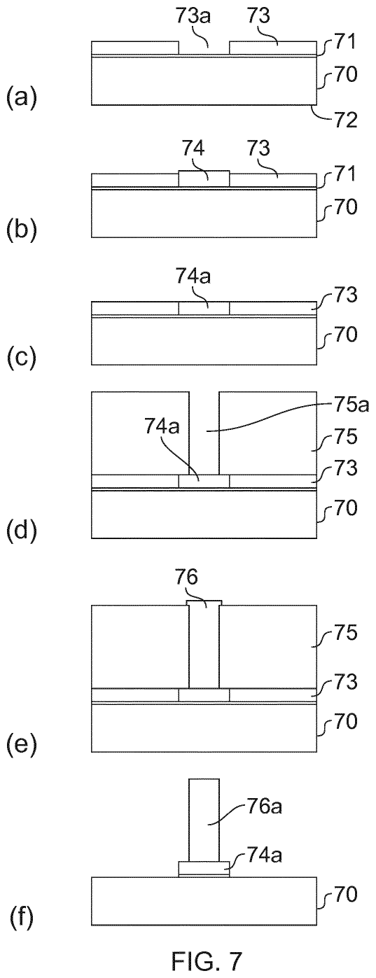

FIG. 7 is a set of schematic cross-sectional views depicting a process flow for fabricating a microelectrode array;

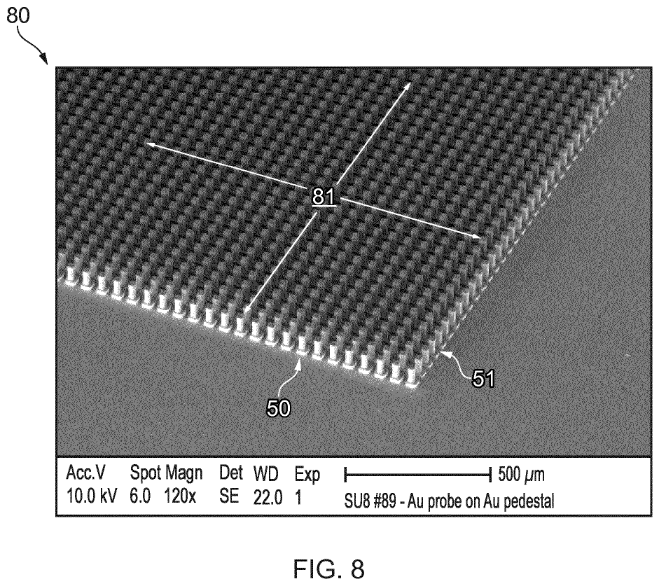

FIG. 8 is a scanning electron micrograph showing a microelectrode array fabricated using a process described herein;

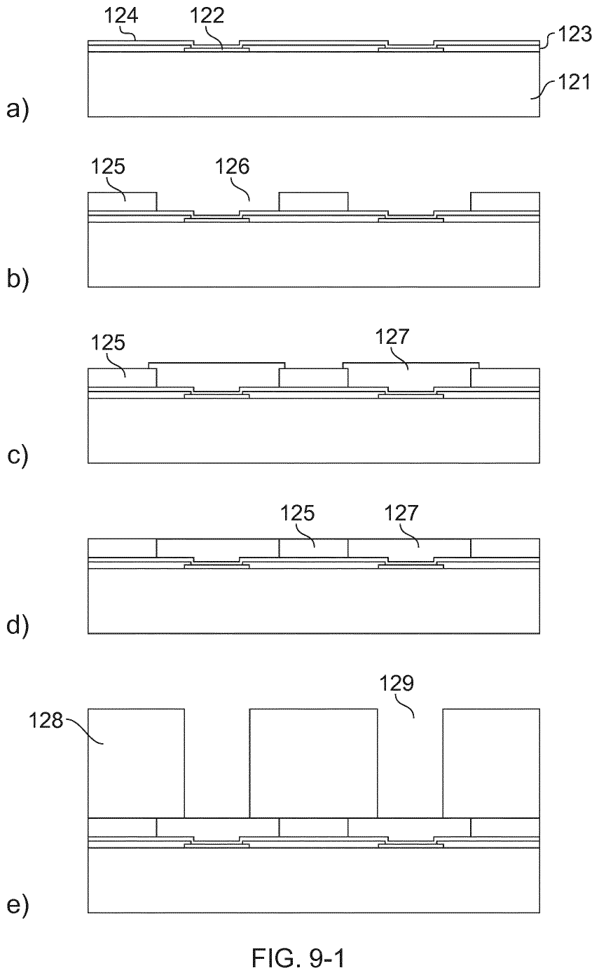

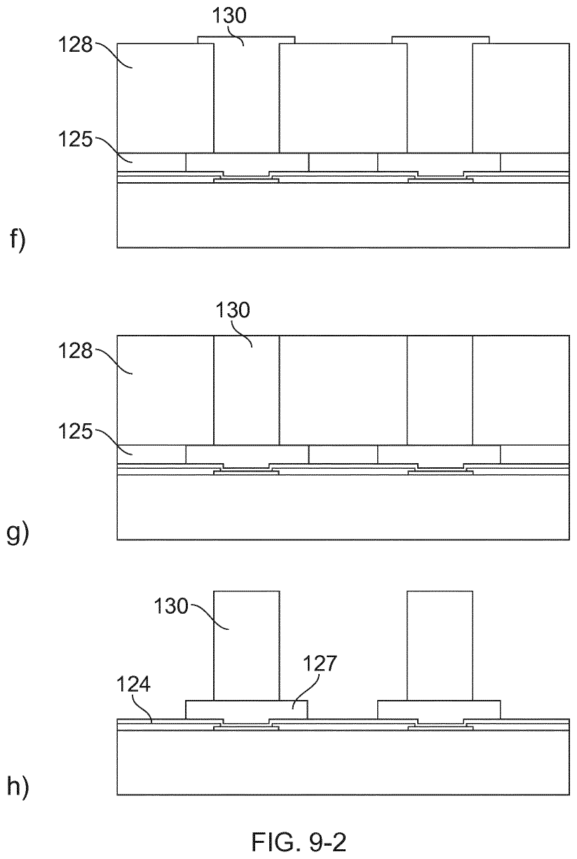

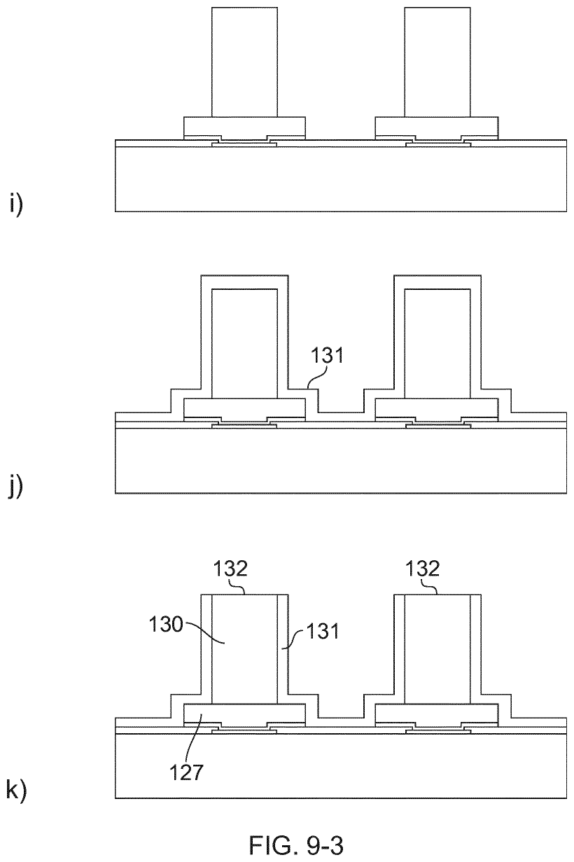

FIG. 9 is a set of schematic cross-sectional views depicting a process flow for fabricating a microelectrode array on an application specific integrated circuit substrate;

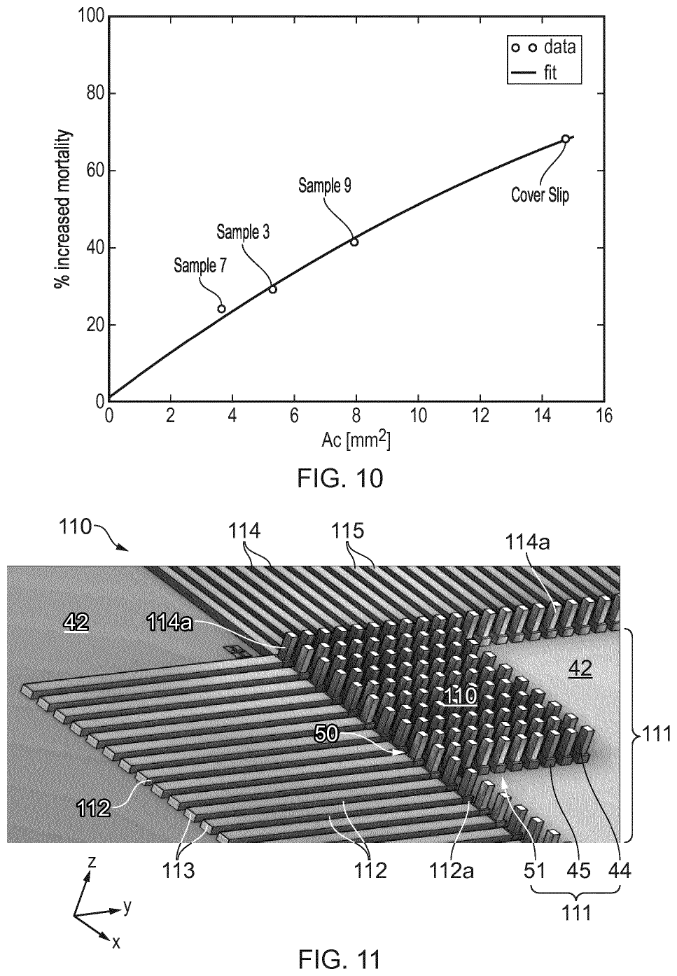

FIG. 10 is a graph showing increases in sample mortality as a function of electrode layout;

FIG. 11 is a schematic perspective view of a microelectrode array with supply channels.

Throughout the present specification, the descriptors relating to relative orientation and position, such as “top”, “bottom”, “horizontal”, “vertical”, “left”, “right”, “up”, “down”, “front”, “back”, as well as any adjective and adverb derivatives thereof, are used in the sense of the orientations as presented in the drawings and/or a mode of use as presented in the drawings. However, such descriptors are not intended to be in any way limiting to an intended configuration or use of the described or claimed invention.

FIG. 3 illustrates schematically in cross-sectional side view a sample structure 20 under analysis engaged with an MEA device 10 having an array of needles 24 penetrating the sample structure. The sample 20 may be any structure under analysis suitable for penetration by the plurality of electrode needles 24 for sampling electrical activity of the sample, and/or stimulating electrical activity in the sample, and/or performing any other kind of electrochemical sensing and actuation. An issue that can arise is controlling the depth or extent of penetration of the needle array into the sample 20, which dictates a spacing or separation 21 of the lower surface 25 of the sample 20 from the substrate 22. In some cases, or in some areas of the sample 20, this spacing 21 may reduce to zero, i.e. implying contact of the sample 20 with the substrate 22. A small spacing 21 may severely reduce or eliminate the possibility of fluid flows to the underside 25 of the sample. Fluid flows to the underside of the sample 20 may be important for a number of reasons, such as oxygenation of the sample, particularly e.g. for thick tissue samples 20, delivery of nutrient to the sample 20, removal of metabolic waste from the sample 20, or homogeneous diffusion of compounds to be tested during screening assays. Such fluids may be referred to herein as critical fluids. Control of the spacing 21 between the underside 25 of the sample 20 and the substrate 22 may also be important for ensuring reproducibility of experimental results.

One way of increasing fluid flows to the underside 25 of the sample 20 is to increase the spacing s or pitch p of the needles 24 thereby allowing wider channels for fluid flow between needles 24. However, such an approach then results in reduced density of needles 24 and therefore a reduced electrode density within the sample 20. This may result in a reduced spatial resolution of data points from the sample 20. Reducing density of the needles 24 may also result in the sample being inadequately supported so that it is difficult to control the spacing 21.

The present invention enables enhanced fluid flows to the underside 25 of the sample 20 while also enabling the maintenance of, or increase in, needle density for a high spatial resolution of data collection points from within the sample 20 under analysis. A microfluid channel system is provided proximal to the base of the needles 24 which may provide for any one or more of improved oxygenation of the sample, e.g. for thick tissue samples, delivery of nutrient to the sample, removal of metabolic waste from the sample and a more homogeneous diffusion of compounds to be tested during screening assays. This may provide an improved experimental setup for reproducibility and automation and decrease time-to-result, e.g. increased throughput. The microfluidic channel system of the invention may also provide for a greater degree of control of the distance of penetration of the needles 24 into the sample 20.

FIG. 4 shows a scanning electron micrograph illustrating in cross-sectional view of a number of needles or pillars 44 of an electrode array 40. In this arrangement, each needle comprises an upstanding pillar or post-like structure extending upwardly from a corresponding pedestal 45. The pedestals 45 are disposed on a substrate 42. Each pillar 44 has a pillar cross-sectional area through the pillar in a plane parallel to the plane of the substrate, and each pedestal 45 has a pedestal cross-sectional area through the pedestal in a plane parallel to the plane of the substrate, with the pedestal cross-sectional area being greater than the pillar cross-sectional area. In this way, the pedestals 45 can each define an upward facing exposed surface 46 substantially parallel to the plane of the substrate 42.

The expression ‘pillar’ as used herein is intended to encompass needle- or post-like upstanding probe structures which have an elongate body of any suitable cross-sectional shape parallel to the plane of the substrate, including square, rectangular, round, rounded, oval or multi-sided. The pillars 44 may have a uniform cross-sectional area all the way up the length of the pillar (or along a substantial part thereof) and may have a flat top, or a profiled top such as a tapered top or a pointed top. Alternatively, the pillars 44 may have a tapered cross-section towards the top or the bottom of the pillar. The pillars 44 are fabricated to be generally suitable for penetrating a sample 20 under test such as a tissue structure or dense neuronal assembly. The expression ‘pedestal’ as used herein is intended to encompass upstanding platforms or plinths with generally planar top surfaces onto which the pillars may be formed or fabricated as will be described hereinafter. The pedestals 45 may preferably have vertical sidewalls or slightly undercut sidewalls as seen in FIG. 4 (tapering inwards towards the base), e.g. according to etch process used.

In the illustrative example of FIG. 4, the pedestals 45 each have a height of about 16 microns and a width of approximately 30-40 microns; the pillars 44 have a height of about 60 microns above the top surface 46 of the pedestal and a width of about 18 microns. In the illustrative example of FIG. 4, the pitch p of the pillars 44 is about 60 to 65 microns, the spacing s is about 40-45 microns and the channel width w_{c} may be about 10 to 30 microns or 14 to 30 microns. The pillars 44 may preferably each be positioned approximately centrally on their respective pedestals 45, though they may also be positioned offset from the centre in one or both orthogonal directions parallel to the plane of the substrate, either deliberately or because of normal photolithography process alignment variability as will be apparent from the illustrative manufacturing processes discussed below.

The spacing w_{c} between each pedestal effectively defines the width of a channel 47 between pedestals 45 in the array of pedestals. As will be evident with further reference to the perspective view of the electrode array 40 of FIG. 5, the channels 50, 51 defined between rows of pedestals 45 may extend in two directions, including longitudinal channels 50 (also referred to herein as ‘y-channels’) extending front to back in the perspective of FIG. 5 and lateral channels 51 (also referred to herein as ‘x-channels’) extending left to right in the perspective of FIG. 5.

In the example of FIG. 5, it can be seen that the cross-sectional profile of both pedestals 45 and pillars 44 is approximately square, though other shapes are envisaged as discussed above. Also as illustrated in FIG. 5, the MEA 40 may be formed such that not every pedestal 45 carries a pillar 44. In the example of FIG. 5, alternate x- and y-direction rows of pedestals have no pillars and thus in this example there are two fluid flow channels 51, 50 in each direction (x and y) between each row of pillars 44. The frequency of pedestals 45 compared to the frequency of pillars 44 may generally be adjusted according to requirements. A reason for requiring fewer pillars 44 than pedestals 45 is that it may be beneficial for certain types of biological samples where it is desired to increase the penetration of the sample 20 by the pillars 44. Having fewer pillars 44 may result in the resistance to penetration being decreased. This arrangement also increases the channel 50, 51 availability between pillars thereby potentially enhancing fluid flows.

As shown schematically in the cross-sectional view of FIG. 6, the pedestals 45 provide a series of exposed upward facing surfaces 46 which provide a platform for supporting the underside 25 of a sample structure 20 which has been penetrated by the pillar electrodes 44 or probes thus preventing the sample structure 20 from reaching the substrate 42 and thereby leaving clear channels 50, 51 between the pedestals 45 for flow of fluids, e.g. to supply fluid to or receive fluids from the sample 20 via the underside 25. In this way, penetration of the pillars 44 into the sample 20 can be controlled while ensuring an optimum fluid flow capability to the underside 25 of the sample 20. The planar top surfaces 46 of the pedestals stop the sample structure from reaching the substrate 42 and allow optimisation of the separation of the sample 20 from the substrate 42, and volume of channel 50, 51 supplying fluid to any desired size. Fluid is able to flow into and through the channels 50, 51 from peripheral edges of the array, e.g. beyond the edges of any sample structure disposed on the array, or via specially configured supply channels as will be described later in connection with FIG. 11. Thus, in a general aspect, the channels 50, 51 are each accessible for fluid flow therethrough from at least two inlet/outlet ends when a sample structure is disposed on the array and in contact with the upward facing surfaces 46 of the pedestal platforms.

In the example of FIG. 6, it is also illustrated that an MEA 40 comprising the pillars 44 and pedestals 45 as described herein may also include an integrated circuit 60 disposed in the substrate 42, e.g. below each pedestal 45. At one or more peripheral edges of the substrate 42, connector pads 5 may be disposed as described previously.

Also as seen in FIG. 6, the pillars 44 may have a cross-sectional area of between 10×10 microns up to 30×30 microns and the pedestals 45 may have a cross-sectional area of between 30×30 microns to about 46×46 microns, or even 60×60 microns. The pedestal heights h may be in the range 10-40 microns and the pedestal spacing w_{c} may be in the range 10-30 microns giving channel 50, 51 cross-sectional areas of between 10×10 microns up to 40×30 microns, e.g. each channel having a cross-sectional area of at least 100 square microns for flow of fluids therethrough when a sample structure is penetrated by the pillar electrodes and supported on the platform defined by the pedestal. The pedestal heights h may be in the range 10-40 microns and the pedestal spacing w_{c} may be in the range 14-30 microns giving channel 50, 51 cross-sectional areas of between 10×14 microns up to 40×30 microns, e.g. each channel having a cross-sectional area of at least 140 square microns for flow of fluids therethrough when a sample structure is penetrated by the pillar electrodes and supported on the platform defined by the pedestal. Other dimensions are possible according to requirement. The pillars may have heights from, e.g. 50 to 150 microns, or even higher. In some arrangements, an array 40 may have pillars 44 of different heights as seen in FIG. 6A. Such arrangements enable the sample 20 to be sampled at different heights (z-positions) within the thickness of the sample, as well as at different x, y positions corresponding to the pillar positions on the substrate. Although only two different pillar heights are shown in FIG. 6A by way of example, the pillar heights may generally vary across the array in any permutation required.

Various fabrication methods for manufacturing the MEAs are possible. FIG. 7 illustrates one such method. A suitable substrate 70 may be a 150 mm, 200 mm or 300 mm diameter and 700-micron thickness wafer, e.g. of silicon or some other suitable material, though other substrate sizes and materials (such as glass) may be used. The substrate, e.g. wafer, may include integrated circuits 60 (as described in connection with FIGS. 6 and 6A), e.g. for driving/reading the electrodes. With reference to FIG. 7a, after a suitable cleaning process, an oxide layer 71 is formed on the surface of the substrate, e.g. a thermal oxide grown from the e.g. silicon substrate. Alternatively, an oxide deposition process may be used. A metallic layer 72 may be deposited on the underside of the wafer substrate, e.g. by sputtering or by other suitable deposition method. The metal layer 72 may comprise plural layers such as 20 nm Ti/300 nm Cu/20 nm Ti. A photoresist layer 73 is deposited and patterned using a suitable lithography process to leave apertures 73a in the photoresist suitable for defining pedestals therein. The photoresist 73 may therefore be of any suitable thickness for forming the pedestals, e.g. 15 microns. The photoresist may be of any suitable type, e.g. SU-8 3000 negative resist.

As shown in FIG. 7b, the wafer is then plated to a first depth, e.g. with an 18-micron gold layer 74 within the apertures 73a for the formation of pedestals 45. Other metal deposition processes may be used for forming the layer 74. The gold layer 74 may then be reduced by a diamond turning process to a second depth, e.g. to 14 microns to planarize the top and form the pedestals, 74a (FIG. 7c). A further layer of photoresist 75 (e.g. 90 microns) is deposited on the first photoresist layer 73 and is patterned using a photolithographic process to form apertures 75a that are co-registered with the pedestals 74a and suitable for forming the pillars 44 (FIG. 7d). A further gold plating process is used to deposit the pillar material 76 (FIG. 7e). Other metal deposition processes may be used. Finally, the gold layer 76 is diamond turned to reduce the height, e.g. to 80 microns, to planarize the top surface and form the pillars 76a. The resist layers 75, 73 are then stripped, the exposed oxide layer 71 removed and the underside metallization etched away leaving the pedestal and pillar structures as shown in FIG. 7f. Although the preferred planarization processes have been described as diamond turning processes as this may enhance adhesion of subsequent layers, other methods of material removal may be possible, e.g. other mechanical machining processes or non-selective etching processes.

With this processing technique, an electrode array 80 of pedestals and pillars can be formed in a two-dimensional grid area 81 (only part of which is shown) with channels 50 and 51 extending between the pedestals, as seen in FIG. 8. This exemplifies an arrangement in which the pedestals and pillars are formed of the same electrically conductive material, e.g. gold, although other materials may be considered such as platinum and iridium, and alloys of gold, platinum and/or iridium.

Another fabrication process is described with reference to FIG. 9 for fabricating an electrode array on a prefabricated application-specific integrated circuit (ASIC). FIG. 9a shows an ASIC 121 defining a substrate with the metal contact pads 122 of the ASIC used for electrical contact to the electrodes of an array to be defined thereon. Passivation (e.g. oxide) layer 123 is opened over the contact pad 122 of the ASIC 121. The contact pad may be aluminium. After removing the oxide 123 over the Al contact pad 122 as seen in FIG. 9a, by using a back etching process for example, the IC 121 is covered with an electrochemical seed layer 124 which may comprise a first adhesion metal, e.g. TiW and a conduction layer, e.g. copper.

As seen in FIG. 9b, a photoresist 125, e.g. SU8, with a thickness in the range of 10-40 microns, is spun and patterned in order to create a mold with openings 126 for pedestal electro-plating. As seen in FIG. 9c, the mold thus created is electroplated with gold 127. The gold layer 127 forms the pedestals. The wafer is then planarized, e.g. using a diamond turning process to result in the structure of FIG. 9d. With reference to FIG. 9e, a further photoresist layer 128, e.g. SU8, with a thickness in the range of 50-150 microns, is spun and patterned in order to create a mold with openings 129 for pillar electro-plating. The mold thus created is electroplated with gold 130 as seen in FIG. 9f. The gold 130 provides the pillars. The resulting structure is then planarized, e.g. by using a diamond turning process to result in the structure of FIG. 9g. The photoresist molds 128, 125 are then stripped (FIG. 9h) and the electrochemical seed layer 124 is etched to remove an electrical short between the probes resulting in the structure of FIG. 9i. The structure is then covered with an electrical insulation coating 131, e.g. by vapor deposition of Parylene (FIG. 9j). Using a planarization process, e.g. diamond turning, the insulation coating 131 is removed from the top of the probe to leave electrodes with an electrical sensing area 132 located on the top of the pillar only.

The fabrication processes described above may include the deposition of a suitable layer of electrical insulation material over the electrode structures comprising the pedestals 45 and pillars 44 and then subsequent selective removal of parts of the insulation material in selective parts of the pillars 44, e.g. on the top surface and/or proximal to the top surface and/or any parts of the sides of the pillars 44. This exemplifies an arrangement in which the pedestals and a lower portion of each pillar are coated with an electrically insulated material. The insulation material may be provided as a conformal coating over the entire electrode structure 44, 45, e.g. by way of a chemical vapour deposition (CVD) process as described above in connection with FIG. 9. A suitable insulation material may be parylene. Selective etching of parts of the insulation material coating may then be performed to expose parts of the electrode.

The processes described above may be implemented in conjunction with the fabrication of suitable electronic circuits 60 and conductive paths 3 as described above, e.g. using established monolithic CMOS wafer fabrication processes.

Tissues and 3D cultures placed on a planar array or any planar substrate suffer oxygen deprivation, which distorts any experimental results. In physiological conditions, (e.g. inside our brain), cells are separated from vessels transporting nutrients and oxygen no more than about 100 μm. 3D neuronal cultures and brain slices are typically 300-400 μm thick, thus when they are placed on a planar surface, the cells facing the surface would be too distant from oxygen supply, thus suffering from hypoxia and going into apoptosis. Consequently, the biological preparation degrades quickly and cannot be recorded for prolonged times. Furthermore, diffusion of tested compounds (e.g. in pharmaceutical screening) is not homogeneous and can lead to a non-physiological relevant response of the tissue and by this altering the validity of the result. The electrode arrays 40, 80 deploying the pedestals 45 for defining micro-channels 50, 51 between each electrode greatly improve the ability to deliver fluids conveying oxygen and nutrients or a desired compound to be tested to regions of the tissue or 3D cell culture and thus reach optimum (physiological) distribution.

Tests have shown significant improvements in cell vitality for cells in samples 20 penetrated by the electrode arrays 40, 80 described above having channels 50, 51 compared to electrode arrays without channels 50, 51, as seen in the table below. Vitality was tested 1 hour after placing a tissue sample onto the electrode array. The pillar size is indicated as the width of the pillar 44 respectively in x and y directions; the pedestal side is the width of the pedestal 45 in both the x and y directions; the microchannel 50, 51 side is the channel width w_{c} (FIG. 4). The pillar/pedestal ratio is the proportion of pedestals 45 that bear pillars and CS* indicates comparative samples where the tissue sample 20 was placed directly onto a coverslip flat surface with no microchannel present. Significant improvements in vitality were realised by greater size of microchannels.

The following table shows the percentage variation in mortality comparing the brain tissue surface in contact with the chip/coverslip vs. the other surface of the same sample directly exposed to the liquid. Each slice tested sample covers an area of 200×200 square microns. In the table, A_{c} means area of contact and mortality values are the average of the measured values+/−standard error.

| | Pedestal | μchannel | | | # | # of |
|---|---|---|---|---|---|---|
| | side | side | A_{c} | % increased | tested | samples |
| Type | [μm] | [μm] | [mm²] | mortality (*) | slices | per slice |
| 7 | 30 | 30 | 3.69 | 23.97 +/− 2.84 | 3 | 10 |
| 3 | 36 | 24 | 5.31 | 28.95 +/− 2.30 | 3 | 10 |
| 9 | 44 | 16 | 7.92 | 41.25 +/− 2.95 | 3 | 10 |
| CS | — | | 14.75 | 68.02 +/− 3.93 | 2 | 10 |

FIG. 10 shows a graph of cell mortality as a function of area in contact with the brain tissue and a tentative quadratic fitting curve. The area in contact is defined as the total area of the top surface of the pedestals (including the area on which a pillar may stand). For instance, an array with 4096 pedestals with pitch of 60 μm and with pedestals of 30 μm width, results in a total contact area of 0.03×0.03×4096=3.69 mm². The equivalent contact area for a coverslip is given by the entire area where pedestals and channels between them are placed, i.e. 0.06×0.06×4096=14.75 mm².

The pillar and pedestal arrangement may also assist in controlling a required extent of penetration of the electrodes into the samples, reaching beyond any dead cell layers and to a consistent depth position within the sample.

Many modifications may be made to the electrode arrays as described above. Delivery of fluids to the channels between pedestals may be enhanced by the provision of supply channels that are configured to direct fluid to the channels 50, 51 that are disposed between the pedestals. As seen with reference to FIG. 11, an electrode array 110 (which is schematically illustrated only in part) defines a two-dimensional grid area 111 in x-y space, i.e. parallel to the plane of the substrate 42, in which the pillars 44 extend upwards in the z-direction on pedestals 45. A first set of channels 50 extend in the y-direction along the plane of the substrate 42 and between the pedestals 45. A second set of channels 51 extend in the x-direction along the plane of the substrate 42 and between the pedestals 45. Outside the periphery of the two-dimensional grid area 111 are provided a first set of supply channels 112 which extend between elongate lands 113 and communicate at distal ends 112a with respective ones of the channels 50 between the pedestals 45 inside the two-dimensional grid area 111. Similarly, outside the periphery of the two-dimensional grid area 111 may be provided a second set of supply channels 114 which extend between elongate lands 115 and communicate at distal ends 114a with respective ones of the channels 51 between the pedestals 45 inside the two-dimensional grid area 111. These supply channels 112, 114 may extend in the respective x- and y-directions, though they need not be straight, nor need they be in relatively orthogonal (x-y) directions to one another.

Similarly, the channels 50, 51 need not be orthogonal to one another, e.g. if the pedestals 45 are not rectangular in cross-sectional profile, e.g. if they are triangular or diamond shape. In the case of triangular cross-sectional profile pedestals, it can be understood that more than two intersecting sets of channels 50, 51 may be provided, e.g. three sets of channels at relative angles of 60 degrees.

In further arrangements, it would be possible for the channels 50 to extend only in one direction across the two-dimensional grid area. Such a construction could be realised by providing the pedestals as elongate structures extending partially or fully across the entire two-dimensional grid area forming a one-dimensional array with channels therebetween. Each pedestal would thereby be an elongate structure carrying a linear row of pillars extending upwardly therefrom. In this way, fluid flows through the channels between pedestals may be confined to non-intersecting channels.

Arrays of 3D MEAs or probe arrays, as described above, each with their associated electronic integrated circuits 60 and connector pads 5 may be formed on a single wafer, typically hundreds of such MEAs which may be diced and packaged using known integrated circuit manufacturing techniques. Each MEA may include hundreds or thousands of individual electrodes.

Examples of MEAs made in accordance with the techniques described herein may generally have any x- and y-dimension of the pedestal as required and any height or z-dimension of the pedestal as required. Typical pedestal heights may lie in the range 10-30 μm or even 10-50 microns. Similarly, the pillars may generally have any x- and y-dimension as required, and any height or z-dimension as required. Typical pillar heights may lie in the range 80-100 μm, 50-100 μm or even 10-200 μm. Micro-channel dimensions between pedestals may be any required size, e.g. 20×20 μm² in cross-section. Another option is to fabricate pillar extensions on top of pillars to extend the height of the pillar, if required. Tight control of the co-registration of mask layers to enable pillar extension layers to be registered to underlying pillars during the fabrication process may be required.

The dimensions of the pillars and pedestals are preferably adapted to work with different kinds of sample structures or materials. In case of brain tissue, the sample material is very soft and a relatively larger pedestal area supportive of the tissue may be required to prevent the tissue from reaching the substrate when the tissue is engaged with the pillars. By contrast, a relatively smaller pedestal area supportive of the sample may be provided for cells inside membrane scaffolding. The dimensions of the pillars and pedestals may be adapted according to, for example, the stiffness and other material properties of any scaffolding materials in use.

In some aspects, the pillar dimensions may be kept as small as possible to enable easy penetration of the sample while still being able to place on the top of the pillar any required sensor or actuator. One constraint may be the technology chosen for the production of the probes. In some aspects, the pedestal dimensions may be kept as small as possible to enable as large a channel as required or possible, to allow a larger surface area of the sample to be exposed to the fluid in the channels while still being able to prevent the sample from sagging or otherwise coming into contact with the substrate between the probes/pedestals, which would tend to block fluid flow through the channels. As evident from table above showing brain tissue mortality, the mortality increases significantly for diminishing channel size.

The pillar height may also be adapted according to requirement. Taller pillars may be deployed to reach into deeper layers of the sample. A brain tissue sample could be 200 μm thick, in which case a pillar of height 50-60 μm is good. However, brain tissue samples of 400 μm thick may require substantially taller probes.

The pillar and pedestal electrode structures described herein can, as discussed, be used for recording signals from cells and for electrically stimulating cells, for example with electrogenic cells such as neurons and cardiac cells. The electrode structures can also be used for cellular impedance measurements, or for chemical sensing, for instance through functionalization of the electrode surface, to readout ions, metabolites and target molecules such as pH, oxygen, neurotransmitters, proteins, hormones of any kind of cell preparation.

However, in a further arrangement, it may be possible to use alternative sensor types, e.g. in which one or more or all electrodes as described in the foregoing examples can be replaced by, or supplemented with, an optical sensor (e.g. a photodiode) and/or an optical actuator (e.g. at LED) respectively for light detection and light stimulation of the sample 20. Thus, each of the pillar/probe structures described above could be replaced by an alternative (non-electrode) type of probe structure, or have an alternative non-electrode type of sensing structure incorporated within an electrode pillar structure.

In other arrangements, a probe array may be constructed with optical sensors and/or optical detectors mounted separately from the probes penetrating the sample. For example, optical elements and/or other remote sensing/actuation devices could be disposed within one or more of the channels 50, 51, on one or more of the surfaces of the pedestals 45 and rely on remote sensing/actuation of the sample, e.g. by electromagnetic radiation.

In other arrangements, the probe structures may be fabricated without any sensing or actuation structures, merely serving as sample supporting structures with the pedestals and channels serving for fluid supply. Sensing and/or actuation functions may be implemented by way of other devices adjacent to or remote from the probe structure, e.g. above or below the sample on the probe structure.

The probe structures described herein may also be used to obtain measurements from different kinds of three-dimensional biological models such as three-dimensional cell preparations built on scaffoldings for 3D cultures (e.g., nanofiber scaffolds, porous membrane scaffolds, hydrogel), three-dimensional tissue samples such as thick slices from organs, spheroids, organoids, full living organisms such as zebrafish and intact explanted or not explanted organs. Throughout the present specification, the expression ‘tissue’ is used to encompass any of these samples. The expression ‘probe structure’ is intended to encompass any microstructure configured for engagement with and support of such 3D samples or cell preparations on a millimetre or sub-millimetre scale.

Other embodiments are intentionally within the scope of the accompanying claims.

1. An in vitro probe array comprising:
a substrate having an array of pillar probes, each pillar probe extending in a direction orthogonal to the plane of the substrate and having a first cross-sectional area, each probe being disposed on a pedestal formed on the substrate wherein each pedestal has a second cross-sectional area greater than the first cross-sectional area and forms a platform from which the pillar probe extends,
wherein a plurality of channels is formed between the pedestals, the channels each having a width less than the spacing between the pillar probes and wherein the plurality of channels define a microfluid channel system,
wherein each pedestal defines an exposed surface substantially parallel to the plane of the substrate at the base of the pillar extending therefrom and the pedestals provide a plurality of said exposed surfaces configured to provide a platform for supporting, in use, the underside of a sample structure when penetrated by the pillar probes to thereby prevent the sample structure from reaching the substrate and thereby blocking fluid flow through the channels.

2. (canceled)

3. (canceled)

4. The probe array of claim 1, in which the pedestal height is greater than 10 microns and the channels each have a width greater than 10 microns.

5. The probe array of claim 1, in which the pedestals define said channels to each have a cross-sectional area of at least 100 square microns for flow of fluids therethrough when a sample structure is penetrated by the pillar probes and supported on the platform defined by the pedestal.

6. The probe array of claim 1 further including a plurality of pedestals each having no pillar probes disposed thereon.

7. The probe array of claim 1 in which at least some pedestals each have a plurality of pillars disposed thereon.

8. The probe array of claim 1 in which the pedestals and pillars are arrayed in a two-dimensional grid area with a first set of channels extending in a first direction along the plane of the substrate between the pedestals and a second set of channels extending in a second direction along the plane of the substrate between the pedestals.

9. The probe array of claim 8 in which the pedestals are rectangular in cross-section and the first set of channels are orthogonal to the second set of channels.

10. The probe array of claim 7 in which the pillars are arrayed in a two-dimensional grid area and the pedestals form a one-dimensional array with channels therebetween extending across the two-dimensional grid area in one direction, each pedestal supporting a plurality of pillars.

11. The probe array of claim 8, further including a plurality of supply channels each supply channel extending parallel to the substrate outside the two-dimensional grid area and communicating with at least one of the channels within the two-dimensional grid area.

12. The probe array of claim 8 further including:
a first plurality of supply channels each extending parallel to the substrate outside the two-dimensional grid area in the first direction and communicating with at least one of the first set of channels; and
a second plurality of supply channels each extending parallel to the substrate outside the two-dimensional grid area in the second direction and communicating with at least one of the second set of channels.

13. The probe array of claim 1 in which one of:
some or all of the probes comprise electrodes; and
at least some probes comprise an optical sensor or an optical actuator.

14. The probe array of claim 1, wherein some or all of the probes comprise electrodes and wherein the probes comprise pillar electrodes and further comprising an electrically insulating material extending over a major portion of the surfaces of the pedestals and pillar electrodes.

15. (canceled)

16. The probe array of claim 1 in which one of:
the pedestals and pillars are both formed of the same electrically conductive material; and
the pedestals and pillars are both formed of the same electrically conductive material and at least a lower portion of each pillar are coated with an electrically insulating material.

17. (canceled)

18. (canceled)

19. The probe array of claim 1 in which each probe is electrically isolated from other probes and electrically connected to a respective connector pad on the substrate or a respective integrated circuit disposed on or in the substrate.

20. A method of fabricating a probe array comprising:
forming a plurality of pedestals onto a substrate, the pedestals being separated along at least one axis to form a plurality of channels between the pedestals, the channels extending along an axis parallel to the surface of the substrate and transverse to the axis of separation of the pedestals;
forming a plurality of pillar probes on the pedestals, each pillar extending in a direction orthogonal to the plane of the substrate and having a first cross-sectional area,
wherein each pedestal has a second cross-sectional area greater than the first cross-sectional area and forms a platform from which the pillar probe extends, and wherein each of the channels has a width less than the spacing between the pillar probes, and
wherein the plurality of channels define a microfluid channel system, wherein each pedestal defines an exposed surface substantially parallel to the plane of the substrate at the base of the pillar extending therefrom and the pedestals provide a plurality of said exposed surfaces configured to provide a platform for supporting, in use, the underside of a sample structure when penetrated by the pillar probes to thereby prevent the sample structure from reaching the substrate and thereby blocking fluid flow through the channels.

21. The method of claim 20 further comprising the steps of:
forming a first photoresist layer on the substrate to a first depth and defining a first plurality of openings therein;
depositing a first metallic layer in the first plurality of openings to form the pedestals;
forming a second photoresist layer on the first metallic layer and on any residual parts of the first photoresist layer, and defining a second plurality of openings in the second photoresist layer that are co-registered with the first metallic layer within the first plurality of openings, each of the second plurality of openings being smaller in cross-sectional area than each of the first plurality of openings;
depositing a second metallic layer in the second plurality of openings to form the pillar probes;
removing any residual first and second photoresist layers.

22. (canceled)

23. The method of claim 21 in which the deposition of one or both of the first and second metallic layers comprises an electroplating process.

24. The method of claim 20 further including, prior to forming the plurality of pedestals on the substrate, forming an oxide layer on the substrate.

25. The method of claim 20 further including depositing an electrically insulating layer onto a major portion of the pillars and pedestals.

26. The method of claim 25 in which depositing an electrically insulating layer comprises depositing the electrically insulating layer to cover the pillar and pedestal structures and selectively removing parts of the electrically insulating layer on the top and/or sides of at least some of the pillars.
